(19) United States
(12) Patent Application Publication
(10) Pub. No.: US 2008/0154362 A1
(43) Pub. Date: Jun. 26, 2008

(54) "W" ACCOMMODATING INTRAOCULAR LENS WITH ELASTIC HINGES

(75) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C&C VISION INTERNATIONAL LIMITED

(21) Appl. No.: 11/933,090

(22) Filed: Oct. 31, 2007

Related U.S. Application Data
(63) Continuation-in-part of application No. 11/685,675, filed on Mar. 13, 2007.
Application No. 11/685,675 is a continuation-in-part of application No. 11/620,488, filed on Jan. 5, 2007.
Application No. 11/620,488 is a continuation-in-part of application No. 11/459,862, filed on Jul. 25, 2006.

(51) Int. Cl. A61F 2/16

(52) U.S. Cl. 623/6.37

(57) ABSTRACT

An accommodating intraocular lens wherein the optic is moveable relative to the ends of extended haptic portions. The lens comprises an optic made from a flexible material combined with haptics capable of multiple flexions without breaking. Each haptic has in longitudinal cross section two wide and deep hinges to better allow the elastic hinges to “stretch” when the optic is subjected to posterior pressure thus allowing the optic to move forward relative to both the outer and inner ends of the haptics. When this movement is combined with the movement of the optic relative to the outer ends of the haptics, the anterior movement of the whole lens, and a change in shape of the optic, the refractive power of the eye is further enhanced.

### CROSS REFERENCE TO RELATED APPLICATIONS

This application is a continuation-in-part of application Ser. No. 11/685,675 filed Mar. 13, 2007, which is a continuation-in-part of application Ser. No. 11/620,488 filed Jan. 5, 2007, which is a continuation-in-part of application Ser. No. 11/459,862 filed Jul. 25, 2006, all of which are incorporated herein by reference.

### BACKGROUND

Intraocular lenses have for many years had a design of a single optic with loops attached to the optic to center the lens and fixate it in the empty capsular bag of the human eye. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990's an acrylic material was introduced as the optic of lenses. The acrylic lens comprised a biconvex optic with a straight edge into which were inserted loops to center the lens in the eye and fixate it within the capsular bag.

Recently accommodating intraocular lenses have been introduced to the market, which generally are modified plate haptic lenses. A plate haptic lens may be referred to as an intraocular lens having two or more plate haptics joined to the optic.

Flexible acrylic material has gained significant popularity among ophthalmic surgeons; however some acrylic materials are incapable of multiple flexions without fracturing. In 2003, more than 50% of the intraocular lenses implanted had acrylic optics. Flexible hydrogel and collamer lenses have also been introduced.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally grooves or hinges can be placed across the plates as part of the lens design to facilitate movement of the optic relative to the ends of the haptics.

### SUMMARY OF THE INVENTION

According to a preferred embodiment of this invention, an accommodating lens comprises a lens with a flexible solid optic attached to which are two or more extended portions. The extended portions, haptics, are plates or loops which can be opened or closed, each haptic capable of multiple flexions without breaking. The haptics preferably have fixation and centration features at their distal ends. The haptics are designed such that upon constriction of the ciliary muscle of the eye with its associated increase in vitreous cavity pressure the haptics move centrally by sliding in the capsular bag pockets. This can be accompanied by making the proximal ends of the haptics adjacent the optic wider than the distal ends. Upon ciliary muscle contraction such a lens design which when placed in the capsular bag causes the plate haptics to move centrally and posteriorly with an increase in vitreous cavity pressure. The haptics have two hinges or groves across each haptic to allow end to end compression of the haptics and facilitate the movement of the ends of the haptics centrally. This causes the proximal end of the plate to move both centrally and posteriorly further increasing the vitreous pressure in addition to that caused by ciliary muscle contraction. The flexible hinge of the haptics preferably have a wide base adjacent the optic separating the sides of the V hinge to make a trough instead of a small V-shaped groove. The distal end of the plate then moves centrally and anteriorly, and with the increase in vitreous pressure and the optic herniates forward by stretching of the wide elastic hinge base. The wide base of the flexible hinge thereby allows stretching of the elastic base in the longitudinal axis of the lens with ciliary muscle contraction and an increase of vitreous cavity pressure, allowing anterior movement of the optic relative to both ends of the haptics.

In the human, the whole crystalline lens moves forward upon ciliary muscle contraction, which also occurs with accommodating lenses. During ciliary muscle contraction the vitreous pressure increases and this can move the optic of a flexible accommodating lens forward relative to both the proximal and distal end of the haptics.

In addition, with constriction of the ciliary muscle and relaxation of the zonules, the peripheral radial pull on the lens is reduced and the fibrosed capsular bag can then exert a central radial longitudinal force on the lens which can cause a change in shape of the optic such that, in addition to optic movement, it adds power to the change in the eye's refraction. This can occur by either deformation of the haptic or by an increase in the thickness of the optic center with a decrease in its radius of curvature.

The accommodating power of the accommodating IOL upon ciliary muscle contraction can therefore be the combination of three factors; namely:

a) The anterior movement of the whole lens since that occurs in the human crystalline lens.

b) An increase in vitreous cavity pressure that causes the posterior vaulted haptics in the eye to move centrally and the lens to assume a “W” shape, thereby allowing the posteriorly vaulted lens optic to change the angle between it and the two haptic components on each plate haptic and to move forward relative to both the outer and inner ends of the plate haptics.

c) Deformation of the thin lens optic.

The various mechanisms can act alone or in combination and are mainly dependent on the design of the haptics. The preferable design is a plate. The plates may have protrusions on their anterior or posterior surface or on both surfaces.

In some embodiments the haptic sides are parallel, thereby allowing them to slide along the capsular bag pockets upon constriction of the ciliary muscle and relaxation of the zonules. The vitreous pressure pushes on the intraocular lens thereby flattening the posteriorly vaulted lens to move the optic forward relative to the outer ends of the haptics. The bag with its slack zonules is then deformed in the long axis of the lens.

The plate haptics may have parallel sides; however, when the distal ends of the plate haptics are wide. This gives a wider area of contact of the capsular bag pocket with the haptics and stabilizes the lens to give a more predictable distance vision. The narrow proximal end adjacent to the optic when it has a hinge, presents a less resistant hinge base. The hinge base, between the two walls of the hinge, may be widened as noted above to allow it to stretch like an elastic band. The shape of the plate haptic is wider adjacent to the optic allows easier movement centrally of the plate. Since the haptic itself is flexible and elastic, it too can stretch to allow additional anterior movement of the optic.

Accordingly, features of the present invention are to provide an improved forms of accommodating lenses.

### BRIEF DESCRIPTION OF THE DRAWINGS

FIG. 1 is a front elevational view of a preferred embodiment of the present invention.

FIG. 2 is a side view showing two hinges on the plate haptics.

FIG. 3 is a detail view of a hinge with a widened hinge base.

FIG. 4 shows a standard lens.

FIGS. 5 and 6 show the present lens with a wide hinge base and additional hinges to allow the lens to move to a “W” shape.

FIGS. 7 through 14 show variations of the haptics.

FIG. 15 shows an alternative lens embodiment.

According to the present invention, the optic is of a foldable, flexible silicone, acrylic, collamer, or hydrogel material and the haptic plates are of a foldable material that will withstand multiple foldings and stretchings without damage, e.g., silicone, hydrogel or collamer. Preferably, the ends of the plate haptics have fixation elements, preferably T-shaped devices that are attached to the distal ends of the plates which are hinged to the optic by two hinges in each plate haptic.

### DESCRIPTION OF THE PREFERRED EMBODIMENT

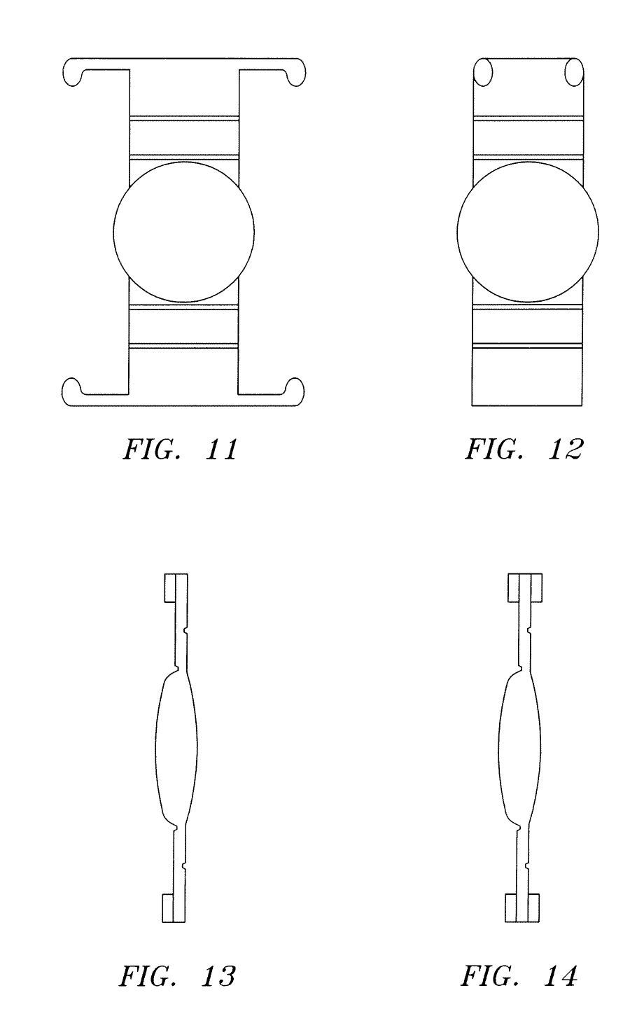

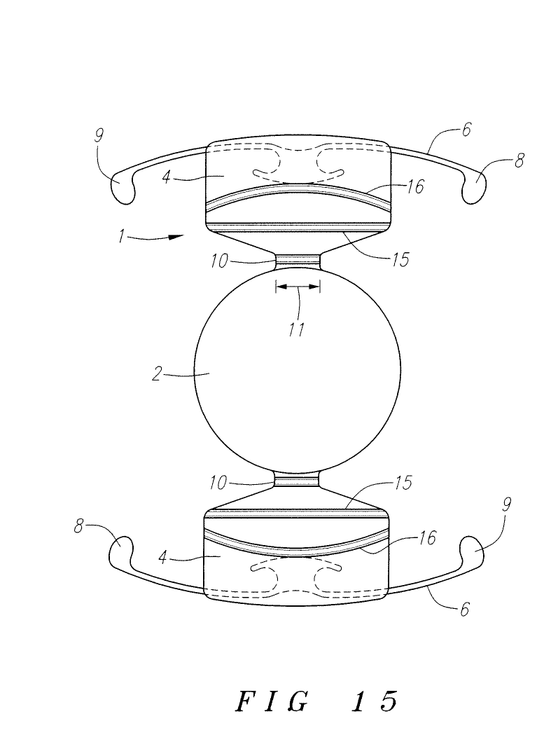

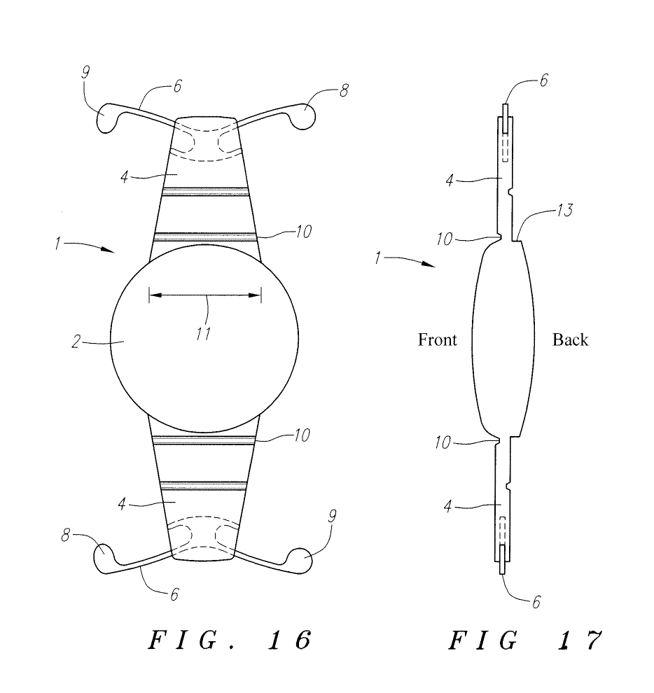

Turning now to FIG. 1, a preferred embodiment is illustrated in detail comprising an intraocular lens 1 formed with a flexible solid optic 2 preferably made of silicone, and flexible extending portions 4 of any suitable form but preferably are silicone triangular plate haptics with narrow or wide bases adjacent to the optic or with parallel sides and which are capable of multiple flexations without damage. The optic 2 and haptics 4 preferably are uniplanar until implanted into the eye, and two or more haptics 4 extend distally from the optic 2. The haptics may be plates, loops or closed loops, and each haptic has two hinges across the plates. Fixation and centration fingers 6 as seen in FIG. 1, or loops or protuberances as seen in FIGS. 9-11 are provided at the distal ends of the hinged haptics 4.

A typical length for the lens is 10.0-12.0 mm, and the optic 2 typically is a 4.5-6.0 mm diameter optic. The fingers 6 preferably are approximately 5.0 mm wide and comprise four-point fixation loops 8 and 9 that flex when the lens is put into any insertion cartridge. The two ends of the four-point fixation loops have a slightly different configuration and aid in indicating to the surgeon that the lens is right side up with the hinges in a proper position.

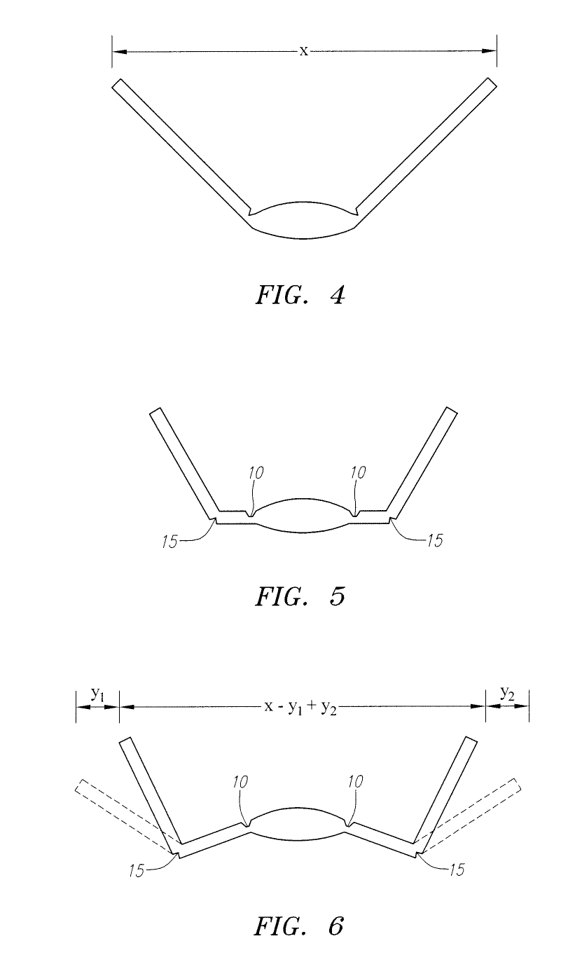

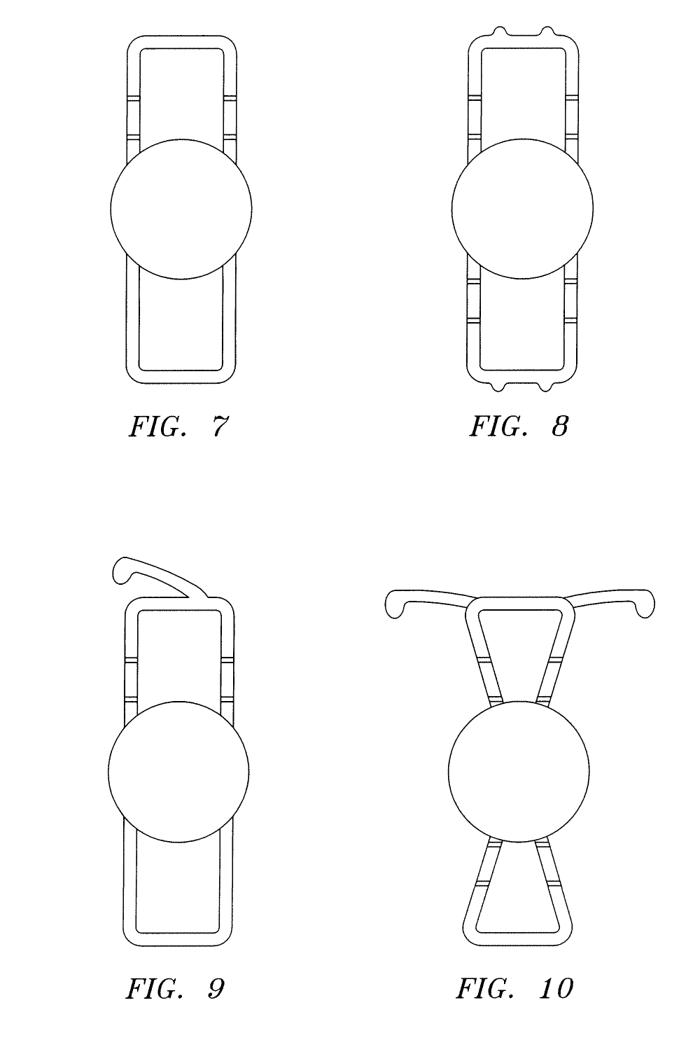

The lens has wide elastic bases 10 to the hinges adjacent the optic such that they can stretch like a rubber band to allow the optic to move by flexion of the two hinges 10, and stretching of its wide elastic base along with a second set of hinges 15 distal to the optic and preferably V-shaped. The hinges allow the plate haptics to assume a “W” shape in side view as seen in FIGS. 5 and 6 with anterior movement of the optic.

The haptics 4 may have a triangular shape, narrower adjacent to the optic, and wider at the outer ends. Two hinges 10 are provided between the haptics 4 and the outer periphery of the optic 2, and it is particularly desirable to have a wide elastic base 12 as seen in FIG. 3 to the narrow hinge width 11 tangential to the optic to further allow the optic 2 to move forward more by stretching of the thin elastic hinge base 10 with the increase in vitreous cavity pressure which allows more anterior movement of the optic than in current designs such as for example that shown in U.S. Pat. No. 6,398,126. A typical hinge width 11 is 1.0-3.0 mm, and preferably with a hinge base width longitudinally as indicated at 12 of 0.06-0.4 mm, and preferably 0.12 mm, as seen in FIG. 3. The hinge base thickness 14 is from 0.5 to 1.5 mm. The wider hinge base 12 stretches like an elastic band to facilitate greater anterior movement of the optic 2. The hinges may be on either the anterior or posterior side of the haptics.

The hinges 10 adjacent to the optic are on the anterior side of the lens and the round end 8 of loops 6 on the right as seen in FIG. 1 indicates that the proximal hinge, next to the optic, is posterior. End 8 is round whereas the end 9 on the opposite loop is oval. The wider loops 6 and wide peripheral plates stabilize the lens and therefore provide better and more predictable distance vision.

Preferably the optic and plate haptics are silicone and the loops 6 are polyimide.

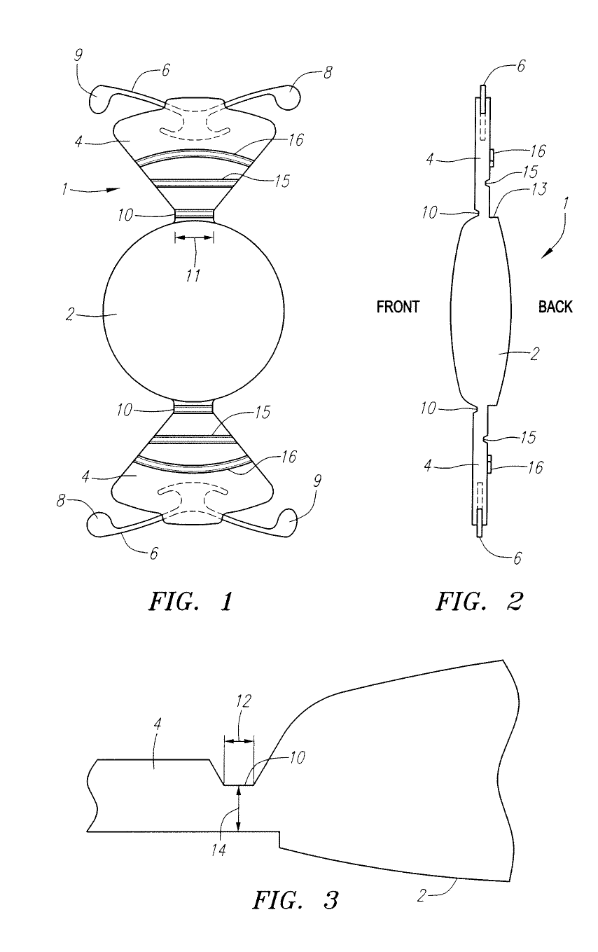

There can be a sharp 360-degree edge 13 around the posterior surface of the optic 2 to reduce the migration of cells across the posterior capsule of the lens postoperatively and thereby reduce the incidence of posterior capsular opacification and the necessity of YAG posterior capsulotomy. There may also be one or more ridges 16 as seen in FIGS. 1 and 2 across the plate to further prevent posterior capsular opacification.

As is well known in the art, the intraocular lens 1 such as that in the drawings is implanted in the capsular bag of the eye after removal of the natural lens. The lens is inserted into the capsular bag through a generally circular opening torn in the anterior capsular bag of the human lens after passing through a small opening in the cornea or sclera. The outer ends of the haptics 4, or loops 6, are positioned in the cul-de-sac of the capsular bag. The outer ends of the haptics, or the loops, are in close proximity with the bag cul-de-sac, and the loops are deflected centrally to conform with the inner surface of the capsular bag. The ends or knobs of the loops are provided on the outer end portions of the loops 6 for fixation to secure the lens in the capsular bag or cul-de-sac with fibrosis, which develops in the capsular bag following the surgical removal of the central lens cortex and nucleus.

The inner ends of the loops 6 may be either integrally formed from the same material as the haptics 4 or the loops may be of a separate material such as polyimide. The loops, if formed of a separate material, are molded into the terminal portions of the haptics 4 or if the lens is lathe cut, attached after the lens body is fabricated.

FIGS. 4-6 show the mechanism of the action of the “W” haptic accommodating lens. FIG. 4 illustrates a standard lens with haptics and hinges adjacent the optic for comparison purposes with FIGS. 5 and 6. FIG. 5 illustrates the lens of the present invention as it would be in vitro in the distance position. This lens has the present wide hinges 10 adjacent the periphery of the optic, and further has the “V” shaped hinges 15 spaced a distance from the optic. FIG. 6 illustrates the lens body moved forward and the haptics moved centrally and posteriorly. Posterior movement of the haptics adds to the increased vitreous pressure with accommodation. The wide hinges 10 essentially are elastic hinges which allow the optic to move forward with an increase in vitreous cavity pressure by elongation or stretching of the base of the hinge 10.

Accordingly, there has been shown and described a lens that ideally comprises a silicone optic and silicone haptic plates with loops at their distal ends that can be of a different material than the plate, and provide fixation and centration of the lens in the eye. The haptics are designed for encapsulation in the tunnel formed by the fusion of the anterior and posterior capsules of the human capsular bag. The lens has wide elastic bases 10 to the posterior hinges adjacent the optic such that they can stretch like a rubber band to allow the optic to move by flexion of the two hinges and stretching of its wide elastic base along with a second set of hinges 15, which preferably are typical “V” shaped hinges. The hinges allow the plate haptics to assume a “W” shape in side view as seen in FIGS. 5 and 6 with anterior movement of the optic.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

What is claimed is:

1. An accommodating intraocular lens comprising a flexible solid optic and attached flexible extended portions comprising double hinged haptics, designed such that they can assume a “W” shape which upon ciliary muscle contraction and move the double hinged haptics centrally and posteriorly such that the optic can move backward and forward relative to the extended portions and whereby the double hinges of each haptic has a thinned and widened elastic hinge base adjacent the optic such that upon an increase in posterior vitreous pressure the thinned and widened elastic hinge areas can stretch like a rubber band so that the haptics can assume a compressed “W” shape to further aid anterior movement of the optic relative to both the outer and inner ends of the haptics.

2. A lens according to claim 1 wherein the haptics are relatively narrow adjacent the optic and are wider distally.

3. A lens according to claim 1 wherein the haptics have parallel sides.

4. A lens according to claim 3 wherein the haptics additionally have two thinned areas on each haptic.

5. A lens according to claim 4 wherein each thinned area is a hinge and is V-shaped.

6. A lens according to claim 1 wherein the thinned area is a hinge and is trough shaped and has a wide base connecting the two sides of a hinge.

7. A lens according to claim 4 wherein the thinned area is a shallow groove.

8. A lens according to claim 1 wherein one or more fixation/centration fingers are on the ends of the extended portions.

9. A lens according to claim 8 wherein the fixation/centration fingers indicate the correct side up of the lens for insertion in the eye.

10. A lens according to claim 8 wherein the fingers are designed to extend beyond the diameter of the capsular bag and are flexible to bend to conform to the bag diameter.

11. A lens according to claim 8 wherein the fingers have a fixation element of a different shape on their proximal ends to enhance centration and fixation of the lens within the capsular bag.

12. A lens according to claim 8 where fingers are made of a different material than the lens, e.g. polyimide, PMMA, Prolene, etc.

13. A lens according to claim 1 wherein the extended portions include loops and/or fixation devices of polyimide.

14. A lens according to claim 1 wherein the lens is made of an optical material or a combination of optical materials that are inert, including silicone, HEMA, acrylic, collamer, or other material.

15. A lens according to claim 14 wherein the lens optic is made of a different material than the haptics.

16. A lens according to claim 1 wherein the optic has a 360-degree square edge on its posterior surface.

16. A lens according to claim 1 wherein the flexible optic is capable of a shape change that increases its refractive power with constriction of the ciliary muscle.

17. A lens according to claim 1 wherein the optic has one or both surfaces that are polyspheric.

18. A lens according to claim 1 wherein the optic has one or more surfaces that are aspheric.

19. A lens according to claim 1 wherein the optic diameter is from 3.5 to 8 mm.

20. A lens according to claim 1 wherein the optic moves relative to the outer ends of the haptics.

21. A lens according to claim 1 wherein the plates have protuberances on either their anterior or posterior sides or both sides.

22. A lens according to claim 1 wherein the extended portions are open or closed loops.

23. A lens according to claim 1 wherein the haptics are plates and have transverse ridges across the posterior surfaces of the plates.

24. An accommodating intraocular lens comprising a flexible solid optic and attached flexible extended portions comprising haptics, wider adjacent to the optic, designed such that the optic can move backward and forward relative to the outer and inner ends of the extended portions and may assume a position such that the optic can be in front of, in the same plane or behind the outer ends of the haptics and can achieve accommodation by the optic moving forward toward the iris, the lens comprising wide and deep hinges in the haptics adjacent the optic, which allows the hinge base to elastically stretch and elongate when the lens optic is subjected to posterior pressure.

25. A lens according to claim 24 where the hinges have a base between the hinge walls that separates one hinge wall from the other.

26. A lens according to claim 24 whereby the lens body is constructed such that upon constriction of the ciliary muscle the haptics move centrally and the proximal ends move posteriorly thereby further increasing vitreous cavity pressure the elastic thin hinge base then stretching to move the optic anteriorly.

27. A lens according to claim 24 where the lens body and optic are silicone.

28. A lens according to claim 24 where the lens body and optic are acrylic.

29. A lens according to claim 24 where the lens body and optic are collamer.

30. A lens according to claim 24 where the lens body and optic are HEMA.

31. A lens according to claim 24 where the extended portions may be integral and of the same material as the optic.

32. A lens according to claim 24 where the extended portions are made of any suitable inert flexible material that is different from that of the lens optic.

33. A lens according to claim 24 where the extended portions include loop and fixation devices of an inert flexible material.

34. A lens according to claim 24 where the optic size is from 3.5 to 6.0 mm.

35. A lens according to claim 24 where the haptics are plate haptics.
